(19) United States
(12) Patent Application Publication
(10) Pub. No.: US 2008/0114309 A1
(43) Pub. Date: May 15, 2008

(54) Acoustic device for needle placement into a joint

(76) Inventor: Stephen D. Zuckerman, Beverly Hills, CA (US)

Correspondence Address: STITES & HARBISON PLLC, 1199 NORTH FAIRFAX STREET, SUITE 900, ALEXANDRIA, VA, 22314, US

(21) Appl. No.: 11/595,981

(22) Filed: Nov. 13, 2006

(51) Int. Cl. A61M 5/00; A61B 17/20; A61N 1/39

(52) U.S. Cl. 604/264; 604/22; 607/1

(57) ABSTRACT

An acoustic device is provided which assists in accurate placement of a needle into a human or animal diarthrodial joint. The device includes a handpiece which mounts a needle assembly including an acoustic transducer assembly. The transducer assembly, which is incorporated into the lumen of the needle, transmits ultrasound pulses from the needle tip into the joint area and receives the ultrasound pulses that are returned. The handpiece is manipulated by the user to guide the needle during placement. The returned ultrasound pulses are processed to determine whether the needle is placed in the joint itself rather than in a location adjacent to the joint and a corresponding output is produced to aid the user in effecting proper needle placement.

### FIELD OF THE INVENTION

The present invention relates to the placement of needles into the joints of humans or animals for medical diagnosis or therapy.

### BACKGROUND OF THE INVENTION

Although the invention is certainly not limited to injection of knee joints, this is one notable application of the invention. The importance of knee joint injection is growing. The injection of long-acting steroid preparations continues to be a mainstay of conservative management for osteoarthritis. The injection of hyaluronic acid preparations has increased, and these preparations now represent an important therapy for osteoarthritis.

Historically, knee joint injections have been performed in the specialty setting, but there is a growing need for primary care providers to inject the knee joint routinely. Many patients with osteoarthritis of the knee are managed by primary care providers until they are candidates for joint replacement. Increasingly, specialists such as orthopedists, rheumatologists, and interventional musculoskeletal radiologists see patients in the later stages of disease.

Most knee joint injections are performed blindly, i.e., without the aid of any assisting device or imaging technology for needle placement. One method involves air insufflation technique to elicit crepitus for blind needle guidance (see Glattes R C, Spindler K P, Blanchard G M, Rohmiller M T, McCarty E C, Block J., “A simple, accurate method to confirm placement of intra-articular knee injection.” Am J Sports Med. June 2004; 32(4):1029-31).

Many primary care providers feel uncomfortable injecting the knee joint blindly, since they have not had the opportunity to practice this procedure in volume. Further, blind knee joint injection can be performed incorrectly even by experienced specialists (see Jackson D W, Evans N A, Thomas B M., “Accuracy of needle placement into the intra-articular space of the knee.” J Bone Joint Surg Am. September 2002; 84-A(9):1522-7). A missed injection can result in depositing drugs into the soft tissues surrounding the knee, such as fat, muscle, or anterior fatpad. Inaccurate injection can deprive a patient of needed therapy, cause complications, and decrease the apparent clinical effect of scientifically proven therapies.

X-ray fluoroscopy is the current standard for the guidance of needle placement for injection. Numerous academic articles have described multiple aspects of fluoroscopically guided needle placement in various joints. Ultrasonography has been used for image-guided injection of joints and bursa (see Naredo E, Cabero F, Palop M J, Callado P, Cruz A, Crespo M., “Ultrasonographic findings in knee osteoarthritis: a comparative study with clinical and radiographic assessment.” Osteoarthritis Cartilage. July 2005; 13(7):568-74). However, these approaches involve the use of commercially available ultrasound imaging devices to visualize the joint space and the needle simultaneously. Several commercially available devices are miniature acoustic/ultrasound devices localized at the tip of a needle or catheter. However, these devices exist for the purpose of intravascular ultrasound imaging (IVUS) of major arteries or for the purposes of ultrasound localization of a catheter into a major vein or artery percutaneously. They do not apply to localization in joints.

Arthroscopy, i.e., the use of optical devices to visualize and treat the knee and other joints, is a routine surgical procedure. Numerous patents discuss methods and devices relating to arthroscopic cannulas, trocars, obturators, guides, arthroscopes and related equipment. For example, a small diameter cannular, trocar, and arthroscope system is described in U.S. Pat. No. 6,695,772 to Bon et al. This system is similar to a very large needle that is to be used in an office setting. Similarly to the needle placement techniques discussed above, arthroscopy systems rely on blind placement of the initial instruments by an interventionalist with extensive manual skills.

### SUMMARY OF THE INVENTION

In accordance with the invention, a device and method are provided which, among other applications, aid in the accurate injections of the knee, in a clinic or similar setting, and which thus are of benefit to both patients and primary care providers. It will be appreciated that although the injection of the knee joint is an important application, the device and method can be used in other applications involving the placement of a needle into a patient including the injection or removal of fluid from any diarthrodial joint, such as the hip, ankle, shoulder, elbow or wrist.

According to one aspect of the invention, there is provided a method for positioning a needle within a patient, said method comprising:

providing a device having a distal end and including a needle including a needle tip disposed at said distal end and an acoustic transducer assembly disposed at said distal end in acoustic communication with the needle tip;

positioning the needle within a body substance of a patient by piercing the skin and soft tissue of the patient;

transmitting acoustic energy from the needle tip into the patient;

using the acoustic transducer assembly to receive acoustic energy returned to the transducer assembly through the needle from the body substance of the patient in which the needle tip is positioned; and

processing the returned acoustic energy to provide a determination of the body substance in which the needle tip is positioned.

Preferably, parameters relating to both the transmitted ultrasound energy and the returned ultrasound energy are processed in providing said determination.

In one preferred implementation, the transmitted and returned ultrasound energy are compared with respect to relative intensity and the delay of pulse-echo ultrasound waveforms. In an advantageous embodiment, these waveforms are brief pulses that are emitted by the transducer, echoed from within tissue, and then received by the transducer. Advantageously, properties of different body substances are used for said determination, and acoustic impedance mismatches at tissue boundaries are used. Beneficially, the determination includes discriminating between body substances selected from the group consisting of connective tissue, muscle, fat, synovial tissue, synovial fluid, and intra-articular connective tissue.

Preferably, the method further comprises displaying an indication of the probability that the needle tip is positioned in an intra-articular space within the patient. Advantageously, the method further comprises repositioning the needle, as needed, until the indication displayed represents an acceptable probability that the needle tip is positioned in the intra-articular space.

In one preferred embodiment, the transmitted ultrasound pulse is produced by a transducer, the returned ultrasound pulse is converted into an electrical signal, and the electrical signal and an electrical signal from a power supply for the ultrasound transducer are processed to provide an input in a parameter estimation process that provides said determination.

Preferably, different indications representing different probabilities are provided to user based on the determination.

Advantageously, the processing includes using the different scattering and absorption properties of different biological tissue as a reference in making said determination.

Preferably, the transmitting of acoustic energy is initiated in response to actuation of a user interface.

Preferably, the device comprises a handpiece and the processing takes place within the handpiece.

According to a further aspect of the invention, there is provided a device for assisting in positioning of a needle within a patient, said device comprising:

a handpiece for manipulation by a user;

a needle assembly mounted on one end of the handpiece, said needle assembly comprising a needle including a needle tip;

an acoustic transducer assembly, mounted on said handpiece and disposed on or adjacent to said needle assembly in acoustic communication with the needle tip, for, in use with the needle inserted in the patient, transmitting acoustic energy from said needle tip into the patient and receiving acoustic energy that is returned through the needle tip to the acoustic transducer assembly from a location within the patient; and

processing means for processing the returned acoustic energy to provide a determination of the location within the patient at which the needle tip is positioned.

In one preferred embodiment, the needle includes a lumen and

said acoustic transducer assembly is supported in a portion of said lumen while permitting fluid flow through the lumen.

Advantageously, the acoustic transducer assembly is at least partially embedded in a support material disposed in a portion of said lumen, and said transducer assembly includes at least one transducer element supported on the lumen adjacent to the needle tip.

In one implementation, the transducer assembly comprises a single transducer for transmitting and receiving acoustic energy. In an alternative implementation, the transducer assembly includes a first transducer for transmitting the acoustic energy and said transducer assembly for receiving the returned acoustic energy. Advantageously, the transducer assembly includes at least one piezoelectric transducer.

In one preferred embodiment, the acoustic transducer comprises at one or more transducer assemblies supported in the lumen.

Preferably, the processing means uses parameters related to both the transmitted and the returned ultrasound pulses in providing said determination. In one advantageous implementation, the processing means compares the transmitted and returned ultrasound pulses with respect to relative intensity and delay. Preferably, the processing means uses properties of different body substances in said determination. Advantageously, the determination by the processing means includes discriminating between body substances selected from the group consisting of connective tissue, muscle, fat, synovial tissue, synovial fluid, and intra-articular connective tissue or discriminating the interface between such body substances arising from acoustic impedance mismatches.

Preferably, the device further comprises readout means for displaying an indication of the probability that the needle tip is positioned in an intra-articular space within the patient.

Preferably, the device further comprises a parameter estimation module, and a power supply for producing an electrical output for powering the acoustic transducer, and the processing means includes means for converting the returned acoustic pulse into an electrical signal, and means for comparing the received electrical signal and said electrical output from said power supply for the acoustic transducer to provide an input to said parameter estimation module.

In one implementation, the processing means comprises an integrated circuit board disposed within the handpiece, and at least part of the processing by the processing means takes place within the handpiece.

Preferably, the processing means uses the different scattering and absorption properties of different biological tissue as a reference in making said determination.

Advantageously, the handpiece includes at least one user interface element and wherein said acoustic transducer assembly transmits acoustic energy in response to actuation of said at least one user interface element.

Advantageously, the processing by the processing means takes place within the handpiece.

According to yet another aspect of the invention, there is provided a device for assisting in positioning of a needle within a patient, said device comprising:

a handpiece for manipulation by a user;

a needle assembly mounted on the handpiece, said needle assembly comprising a needle including a needle tip and a central lumen;

an acoustic transducer device, disposed in or adjacent to said lumen, for, in use with the needle inserted in the patient, transmitting acoustic energy through said lumen so as to be emitted from the needle tip into the patent and receiving acoustic energy from the needle tip that is returned to the needle tip from a location within the patient;

processing means for processing the returned acoustic energy to provide a determination of the location within the patient at which the needle tip is positioned; and

a readout, connected to said processing means, for indicating to a user, based on said determination, a probability of the needle tip being located at a predetermined location in the patient.

Preferably, the readout comprises at least two different light outputs indicating at least two different probabilities that the needle tip is located at said predetermined location.

Preferably, the predetermined location is within an intra-articular space within the patient.

Further features and advantages of the present invention will be set forth in, or apparent from, the detailed description of preferred embodiments thereof which follows.

### BRIEF DESCRIPTION OF THE DRAWINGS

FIGS. 1, 2 and 3 are a side elevational view, a top plan view and a transverse cross-sectional view, respectively, of a portion of an injection needle in accordance with one preferred embodiment of the invention;

FIG. 4 is a cross-sectional view of the needle of FIGS. 1 to 3, incorporating a micrometer scale acoustic transducer assembly;

FIG. 5 is a front perspective view of a removable injection assembly in accordance with one aspect of the invention;

FIG. 6 is a front perspective view of an overall device including an acoustic transducer and needle assembly mounted atop a hand-piece, in accordance with one embodiment of the invention;

FIGS. 7 and 8 are respective views of the hand-piece shown in FIG. 6 incorporating the removable injection assembly of FIG. 5;

FIG. 9 is a block diagram of an electro-acoustic signal processing system in accordance with one preferred embodiment of the invention;

FIG. 10 is a block diagram of a signal preprocessing system in accordance with one preferred embodiment of the invention;

FIG. 11 is a block diagram of a signal detection system in accordance with one preferred embodiment of the invention;

FIG. 12 is a block diagram of a system for estimating values for a bank of matched filters used to detect signals in the embodiment of FIG. 11; and

FIG. 13 is a schematic block diagram of a signal transformation system used in a pulse-echo ultrasound device in accordance with a further aspect of the invention.

### DESCRIPTION OF THE PREFERRED EMBODIMENTS

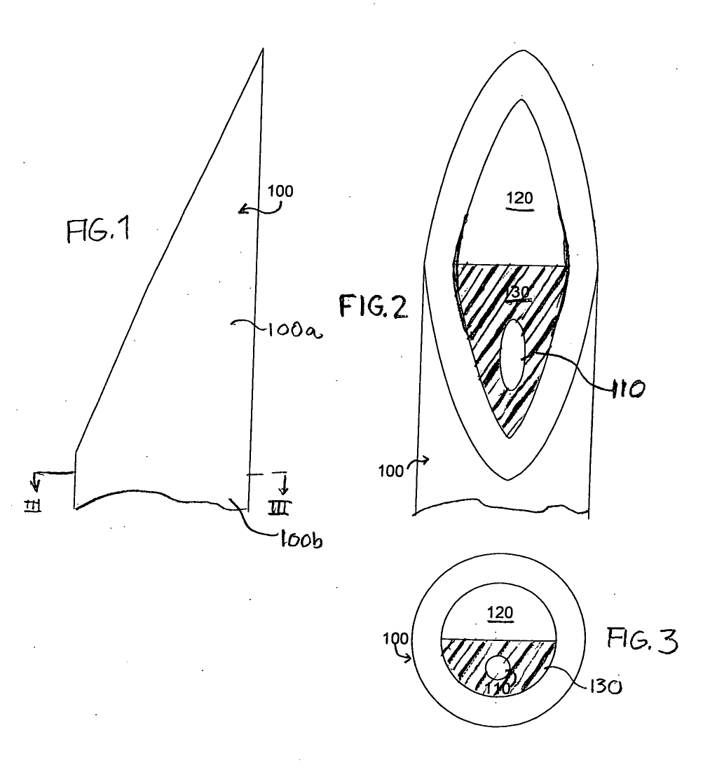

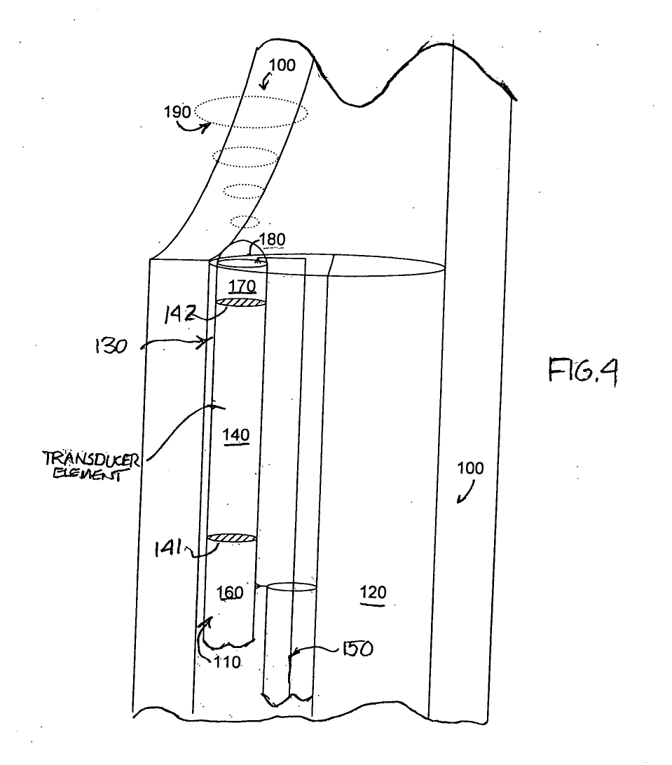

Referring to FIG. 1, there is shown a side elevational view of an injection needle 100 for introducing a medicament into a joint. In the preferred embodiment depicted, the needle 100 is a standard 20G (gauge) needle. Needle 100 includes a sharpened tip 100a and a body or shaft 100b (tip 100a being an inclined or slant portion of shaft 100b). The shaft 100b of the needle 100 is preferably fabricated from medical stainless steel. In the implementation illustrated in FIGS. 2 and 3, one half of the inner diameter of the needle 100 consists of a hollow lumen 120 for injection of medicaments. The other half of the inner diameter of the needle 100 is occupied by a support member 130 preferably comprising a solid epoxy resin. The tip of a micrometer scale acoustic transducer assembly 130 is supported by the solid epoxy resin support member 130. As described in more detail below in connection with FIG. 4, the acoustic transducer assembly 130 has an active element that is located at the tip of the needle. The remainder of the transducer assembly is supported within the shaft of the needle, as illustrated in FIG. 4.

Considering the arrangement illustrated in FIGS. 1 to 3 in somewhat more general terms, a portion of the inner diameter of the shaft 100b of the injection needle 100 is partially occupied by a support element or material 130 such as a solid polymer matrix. A portion of the inner diameter of needle 100 remains and this constitutes a hollow lumen for the flow of fluid medicaments. The solid matrix 130 can be positioned eccentrically or concentrically in order to optimize transducer function. The active element or output of the transducer assembly 130 is preferably located at or proximate to the tip 100a of the needle 100, e.g., at the top of the shaft portion 100b, so that acoustic energy transduced from the active element 180 propagates into the regions surrounding the needle tip 100a.

Referring to FIG. 2, the micrometer-scale acoustic transducer assembly 120 is shown in more detail. Assembly 130 includes a transducer element 140. In an exemplary embodiment, transducer element 140 comprises a piezoelectric crystal element fabricated from fine-grain lead-zirconate-titanate (PZT) ceramic mounted in a cylinder with a diameter of 62.5 micrometers, and a length of 400 micrometers, and has a center frequency of approximately 5 megahertz. The assembly 130 also includes an impedance-mismatching layer 160 and an impedance-matching layer 170. In one implementation, the latter is fabricated from silver-carbon conductive ink with an acoustic impedance of 5MRayl. An acoustic lens 180, preferably fabricated from polyurethane, focuses emitted sound waves, denoted 190, from transducer element 140. A coaxial cable 150 connects transducer assembly 130 to output processing circuitry discussed below. In an exemplary embodiment, coaxial cable 150 has an outer diameter of 125 micrometers and 50 ohm electrical impedance. In the embodiment under consideration, the epoxy resin has an acoustic impedance of 8MRayl. Electrodes 141 and 145 are provided between transducer elements 140 of support member 1 and layers 160 and 170 and each preferably comprises a thin film of nickel.

Considering the embodiment of FIG. 4 in more general terms, the micrometer-scale acoustic transducer assembly 110 is preferably supported by a solid polymer matrix support member 130. The transducer assembly 110 preferably comprises a piezoelectric material with multiple additional layers for mismatching, backing, tuning, matching, and focusing (e.g., lens 180). It will be understood that the geometry of the piezoelectric transducer may be a cylindrical, square, rectangular prism or another geometry in a bar-mode, plate-mode, element-mode (i.e., wherein the poling dimension is larger or smaller than face dimensions). The piezoelectric transducer may be a composite of piezoelectric elements with epoxy or other support elements in 1-3, 2-2 or other geometries. The piezoelectric element may comprise PZT, as noted above, or may comprise other ceramics of lead in coarse or fine grain, PVDF or other polymers, or microelectromechancal system (MEMs) composite transducers. The additional layers may comprise single phase or multiphase solutions or mixtures of mixtures of metals, inks, polymers, ceramics, rubbers, glasses or other substances. The transducer assembly corresponding to assembly 110 may include materials and geometries that support a center frequency from 100 KHz to 100 MHz.

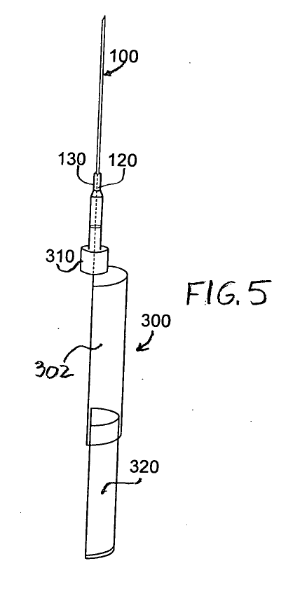

Referring to FIG. 5, there is shown a removable injection assembly 300. Assembly 300 includes a reservoir 302 for injectable substance, and a plunger 320 which creates injection pressure. A molded housing 310 is divided into two halves. One half or side contains a fluidic conduit that transports an injectable substance up the abovedescribed lumen 120 of the needle 100. The other half or side of molded housing 310 is an epoxy filled substrate for electronic and acoustic instrumentation for the abovedescribed epoxy filled half-lumen 130 of the needle shaft or body 100b.

Considering the injection assembly 300 in more general terms, in accordance with the aspect of the invention shown in FIG. 5, a removable injection assembly is provided wherein a needle is integrated into a base. Preferably, a portion (e.g., portion 130) of the needle lumen contains a solid polymer matrix which supports acoustic, electronic, or other components for processing and transduction of energy. In one preferred embodiment, optical components are employed as described in U.S. patent application Ser. No. 11/497,238 filed on Aug. 2, 2006, in the name of Stephen D. Zuckerman. A portion of the needle lumen constitutes a hollow conduit for fluid. This fluid is contained in a reservoir (corresponding to reservoir 302) for an injectable substance. Importantly, the components of the assembly are integrated into a single assembly which is removable from the remainder of the device.

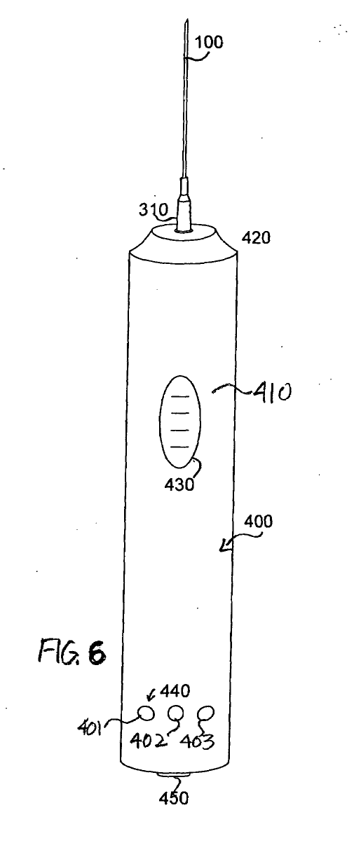

Referring to FIG. 6, there is shown a front elevational view of an overall acoustic transducer assembly and needle assembly corresponding to that discussed above mounted atop a hand-piece 400. More specifically, as illustrated, hand-piece 400 supports housing 310 which, in turn, supports needle 100. Handpiece 400 comprises a cylindrical body 410 having a tapered nose 420 at the distal end thereof and includes a control button 430 for controlling the operation of the device, a readout 440, preferably in the form of three indicating lights or lamps 401, 402 and 403 which preferably comprise light emitting diodes (LEDS), and an external connector 450 for connection to an external power source, processing unit or the like (not shown in FIG. 6), as described below.

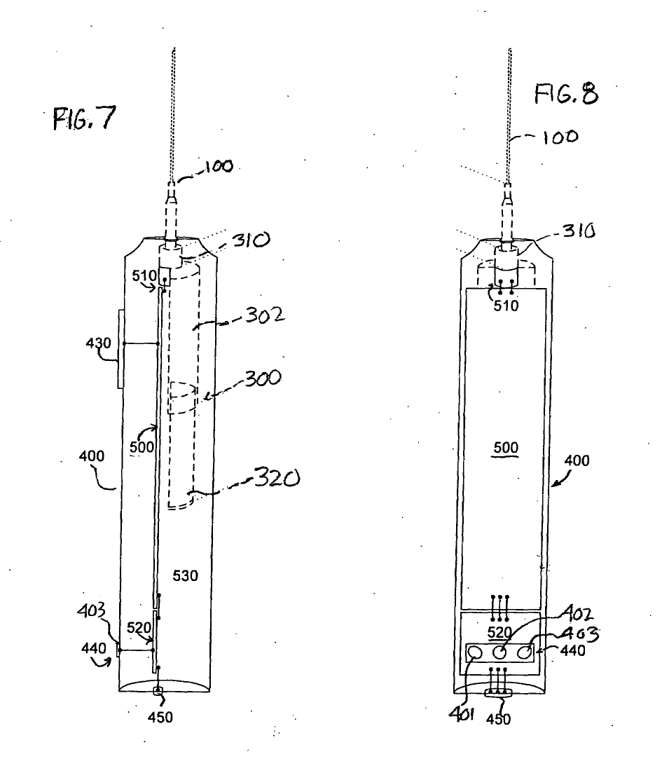

Turning to FIGS. 7 and 8, there are shown two cross sectional views of the cylindrical hand-piece 400 which are rotated 90 degrees from each other. The removable injection assembly 300 of FIG. 5 is shown in dashed lines in FIGS. 7 and 8 and is connected to, and articulates with, an electric coupling 510. The coupling 510 is driven by drive circuitry on printed circuit board 500 which also contains control and signal processing logic, and is connected to control button 430. A further printed circuit board 520 controls the display 440 described above and is connected to the external communication connector or port 450. An additional space 530 is provided in the body 410 of the hand-piece 400 for housing additional equipment such as, for example a removable battery (not shown).

Considering the embodiment of FIGS. 6 t0 8 in somewhat more general terms, a hand-piece such as hand-piece 400 supports needle and transducer assembly such as that described above, and a removable injection assembly such as that of FIG. 5. The hand-piece is used, inter alia, to house connectors which provide electrical connections to the transducer assembly via one or more printed circuit boards, such as boards 510 and 520, which contain conventional control, signal processing, and communication functions and user interface logic. The hand-piece supports one or more user interface elements, such as a control button 430, and a readout such as display 440. A communication connector such as connector 450 is used to support separate system functions.

In operation, in accordance with one embodiment of the invention, the hand-piece 400 is manually manipulated by a skilled user so that the needle 100 atop the hand-piece 400 is inserted through skin and soft tissues of the patient (which can be a human or an animal) into a position tentatively identified by the skilled user as being within, in this example, a diarthrodial joint. The control button 430 is pushed and an electronic test pulse is generated in response. The pulse is transmitted to the acoustic transducer 140, and an acoustic pulse, indicated generally by acoustic pulses 190 of FIG. 4, is emitted proximate to the tip of the needle 100. The acoustic pulse is backscattered by tissue at various depths and a corresponding pulse-echo is received by the acoustic transducer 140 proximate to the tip of the needle 100. The pulse-echo signal is transduced by transducer 140 and the resultant electronic pulse-echo signal is processed as described below. An estimate of the probability that the needle tip is within a joint is produced, and in the embodiment of FIGS. 5 to 8, one of three readout lights 401, 402 and 403 is illuminated to indicate low, medium or high probability.

The hand-piece is iteratively manipulated by the skilled user to vary and improve the position of the needle tip. The button 430 is pressed and the readout observed to gain an indication of the tip position. The process stops when the user is satisfied that the needle tip is in the joint. At this point, injection of the injectable substance, i.e., the substance contained in the reservoir 302 of the removable injection assembly 300, proceeds in response to movement of plunger 320. When injection is completed, the needle 100 is removed from the joint.

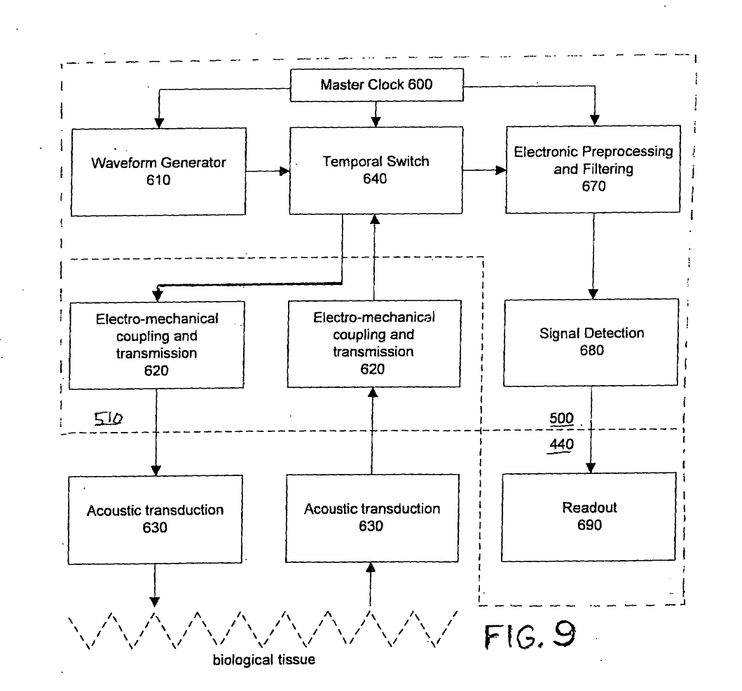

Referring to FIG. 9, there is shown a block diagram of an electro-acoustic signal processing system which can be used for system processing for the device of FIGS. 1 to 8. A pulse waveform generator 610 produces pulses which are generated digitally and undergo digital to analog conversion. An electro-mechanical coupling and transmission function (a coupler and coaxial transmission cable) 620 (which, as indicated, can be mounted on integrated circuit board and corresponds to the circuitry briefly described above). Similarly, an acoustic transduction function (transducer assembly) 630 which can be implemented by the transducer circuit 130 of FIG. 4.

As is indicated schematically in FIG. 9, acoustic energy propagates through tissue and is received by a further acoustic transducer function, which can be the transducer element of acoustic transducer 130 or a separate transducer element. The returned energy is coupled from transduction function 630 to a further coupling and transmission function which, again, can be the same function as the first mentioned function 620 or a separate function.

A temporal switch 640 directs energy between, i.e., switches between, transmission and receipt of acoustic signals. An electronic preprocessing and filtering function 670 provides analog to digital conversion and electronic preprocessing of output signals from switch 640.

A signal detection function 680, which is described in more detail below, detects prototype signals from various tissues types and serves to identify the tissue of origin. The results of the detection operation are displayed by a readout (display) function, which can be implemented by display 440 of FIGS. 1 to 8. The temporal functions of the system are coordinated by a clock 600. Units or functions 600, 610, 640, 670 and 680 are mounted on circuit board 500 of FIGS. 1 to 8, in one embodiment of the invention.

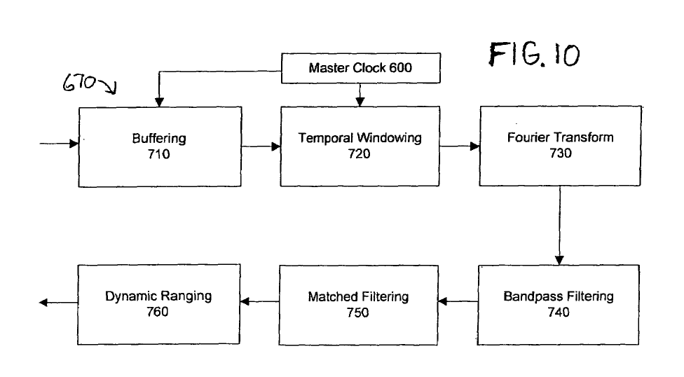

Referring to FIG. 10, there is shown a block diagram of the signal preprocessing function 670 of FIG. 9. The signal initially arrives from a temporal switch controlling the transducer to be latched and is buffered by a buffering function 710. The signal is windowed in time by a temporal windowing function 720 to eliminate early echoes (e.g., transducer artifacts) and late echoes (e.g., deep structures of no interest). The time data is transformed by a Fourier transform function 730 for frequency-domain analysis. A bandpass filtering function 740 provides bandpass filtering around the center frequency of the device so as to eliminate noise. A matched filtering function 750 provides matched filtering of the transmitted waveform so as to improve sensitivity and range. The matched filter function is created by Fourier transformation of the pulse signal from the waveform generator 610 of FIG. 9. A dynamic ranging function 760 provides dynamic range analysis (e.g., a logarithm transform, histogram normalization) so as to ease further processing, given the large dynamic range of acoustic signals.

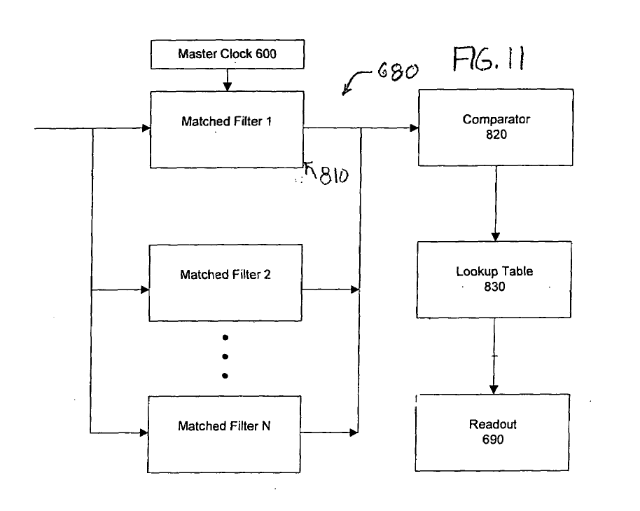

Referring to FIG. 11, there is shown a block diagram of the signal detection function 680 of FIG. 9. A bank of matched filters, denoted 810, receives pulse-echo signals from tissues with a variety of geometries and acoustical properties. The filtered signals produced by filter bank 810 are integrated, and a comparator 820 identifies the filter producing the maximal response. The maximum-output matched filter in the bank 810 is mapped by a lookup table 830 to determine the tissue of origin, and, in particular, to determine the probability that the needle tip is in an intra-articular joint. The lookup table 830 is loaded, e.g., by computer simulation inputs of pulse-echo signals from various geometries with various acoustic properties.

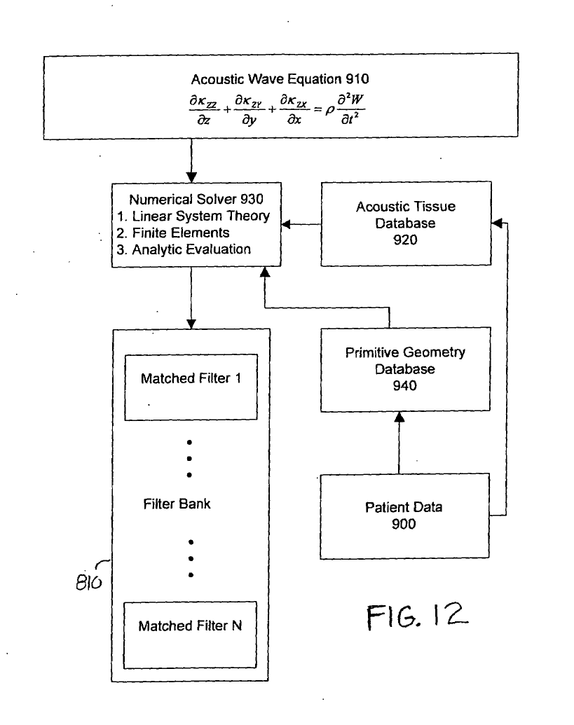

Referring to FIG. 12, there is shown a block diagram for determining the make-up of the bank 810 of matched filters of FIG. 11. As indicated above, the goal is to detect signals when, during positioning the needle, the needle is inside the joint of interest and label these signals as indicating high probability, while rejecting other signals as low probability indicators. A mathematical model 910 for signal propagation is used to simulate signals with a high degree of accuracy through the use of a numerical solver 930. Numerical solver 930 receives inputs from a database 920 of tissue acoustic properties and database 940 of tissue geometries. The content of databases 920 and 940 are derived from a patient database 900 and provide the parameters for the model 910. The model output is stored as frequency-domain data in the form of the bank 810 of matched filters, with one filter for each simulation pulse-echo waveform.

Considering this aspect of the invention in somewhat more general terms, to achieve the stated goal, pulse-echo signals are processed by numerical algorithms to detect signals which indicate that the needle tip is inside the joint of interest whether human or animal. Linear filters, nonlinear filters, wavelet domain filters, artificial neural networks, fuzzy logic systems, nonparametric functional estimation methods, statistical discriminant functions, and other parametric and nonparametric statistical methods are methods used in the art for such signal processing functions. It is to be understood that one or more of these methods may be used in addition to, or instead of, the matched filters approach described above in performing this system function.

As indicated above, matched filters or other numerical algorithms that may be used in the system have adjustable parameters which affect the ability to detect return signals. Such parameters can be adjusted by use of a mathematic model of the signal formation process. In one example, a tissue geometry, particular tissue acoustic characteristics, and a test pulse must be chosen in order to simulate the model. Another approach is to use Monte Carlo methods. Monte Carlo methods involve choosing model parameters at random according to some probability distribution. Other sampling schemes are regular or irregular but deterministic, i.e., require no random choice. Deterministic and Monte Carlo sampling are general methods known in the prior art that may be used for determination of model parameters for simulation of a forward model used to adjust the parameters of numerical algorithms used for signal detection.

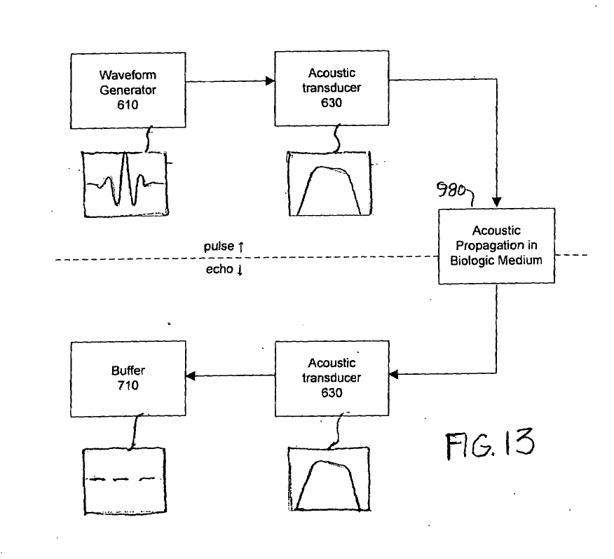

Referring to FIG. 13, there is shown a schematic diagram of the signal propagation in a pulse-echo ultrasound device such as that described above. A pulse waveform of the general characteristics illustrated is produced by a waveform generator (corresponding to waveform generator 610 of FIG. 9 in the preferred embodiment) in order to interrogate the biologic tissue. The pulse-echo signal is transformed by the physics of the associated acoustic transducer (corresponding to transducer 630 of FIG. 9 in a preferred embodiment), and characterized by the power spectrum illustrated. The test pulse propagates through biologic tissue thereby undergoing scattering and absorption, as represented by block 980. A portion of the acoustic energy is backscattered, and is received, and transduced, by transducer 630 and electronically buffered by buffer corresponding to buffer 710 of FIG. 10. As illustrated, the received signal is a temporal sequence of degraded and transformed pulse sequences derived from echoes at various locations and times in the biologic tissue. The backscattering in biologic tissue resulting in detectable echoes is due to acoustic impedance mismatches.

As discussed above, the propagation of acoustic energy in biologic tissue is determined by scattering and absorption. Pulse-echo imaging and detection is an important contrast mechanism in medical ultrasound imaging. Such pulse-echo imaging relies on backscattering and specular reflection at approximately 180 degrees as the contrast mechanism, as described above in connection with the preferred embodiment. However, other contrast mechanisms, pulse sequences, and detection modalities including continuous wave mode, Doppler flow and power modes, and acoustic-radiation-force mode are known in the art and these and other modes of operation may be used in addition to, or instead of, the pulse-echo mode described above. One or more of these embodiments may require the use of a receiving transducer that is distinct from the transmitting transducer.

In another embodiment of the invention, the phenomenon of acoustic-radiation-force is used to augment the pulse-echo mode of operation described above. In this embodiment, pulse-echo data is collected as described above for a preferred embodiment but, in addition, a rapid sequence of pulse-echo interrogations is also undertaken at a rate of approximately 5 kilohertz for a total of approximately 5000 pulses (at the center frequency of 5 megahertz). In this embodiment, displacement of the tissue due to acoustic radiation force contributes to the round-trip temporal delay of pulse-echoes. The increase in successive delays is used to estimate the elasticity or other mechanical properties of tissue through the use of differential equations such as the so-called Voight model (involving a linear mechanical circuit of a spring and dashpot in parallel).

Although the invention has been described above in relation to preferred embodiments thereof, it will be understood by those skilled in the art that variations and modifications can be effected in these preferred embodiments without departing from the scope and spirit of the invention.

What is claimed:

1. A method for positioning a needle within a patient, said method comprising:
providing a device having a distal end and including a needle including a needle tip disposed at said distal end and an acoustic transducer assembly disposed at said distal end in acoustic communication with the needle tip;
positioning the needle within a body substance of a patient by piercing the skin and soft tissue of the patient;
transmitting acoustic energy from the needle tip into the patient;
using the acoustic transducer assembly to receive acoustic energy returned to the transducer assembly through the needle from the body substance of the patient in which the needle tip is positioned; and
processing the returned acoustic energy to provide a determination of the body substance in which the needle tip is positioned.

2. A method as claimed in claim 1 wherein properties of different body substances are used for said determination.

3. A method as claimed in claim 2 wherein said determination includes discriminating between at least two body substances selected from the group consisting of connective tissue, muscle, fat, synovial tissue, synovial fluid, and intra-articular connective tissue.

4. A method as claimed in claim 1 further comprising displaying an indication of the probability that the needle tip is positioned in an intra-articular space within the patient.

5. A method as claimed in claim 4 further comprising repositioning the needle, as needed, until the indication displayed represents an acceptable probability that the needle tip is positioned in the intra-articular space.

6. A method as claimed in claim 1, wherein different indications representing different probabilities are provided to user based on the determination.

7. A method as claimed in claim 1 wherein said processing includes using the different scattering and absorption properties of different biological tissue as a reference in making said determination.

8. A method as claimed in claim 1 wherein the device includes a handpiece and at least part of said processing takes place within the handpiece.

9. A method as claimed in claim 1 wherein said transmitting of acoustic energy is initiated in response to actuation of a user interface.

10. A device for assisting in positioning of a needle within a patient, said device comprising:
a handpiece for manipulation by a user;
a needle assembly mounted on one end of the handpiece, said needle assembly comprising a needle including a needle tip;
an acoustic transducer assembly, mounted on said handpiece and disposed on or adjacent to said needle assembly in acoustic communication with the needle tip, for, in use with the needle inserted in the patient, transmitting acoustic energy from said needle tip into the patient and receiving acoustic energy that is returned through the needle tip to the acoustic transducer assembly from a location within the patient; and
processing means for processing the returned acoustic energy to provide a determination of the location within the patient at which the needle tip is positioned.

11. A device as claimed in claim 10 wherein the needle includes a lumen and said acoustic transducer assembly is supported in a portion of said lumen while permitting fluid flow through the lumen.

12. A device as claimed in claim 11 wherein the acoustic transducer assembly is at least partially embedded in a support material disposed in a portion of said lumen, and said transducer assembly includes at least one transducer element supported on the lumen adjacent to the needle tip.

13. A device as claimed in claim 10 wherein said transducer assembly comprises a single transducer for transmitting and receiving acoustic energy.

14. A device as claimed in claim 10 wherein said transducer assembly includes a first transducer for transmitting the acoustic energy and said transducer assembly for receiving the returned acoustic energy.

15. A device as claimed in claim 10 wherein said transducer assembly includes at least one piezoelectric transducer.

16. A device as claimed in claim 10 wherein said processing means uses properties of different body substances in said determination.

17. A device as claimed in claim 15 wherein said processing means, in making said determination, discriminates between at least two body substances selected from the group consisting of connective tissue, muscle, fat, synovial tissue, synovial fluid, and intra-articular connective tissue.

18. A device as claimed in claim 10 further comprising readout means for displaying an indication of the probability that the needle tip is positioned in an intra-articular space within the patient.

19. A device as claimed in claim 10 wherein said processing means comprises an integrated circuit board disposed within said handpiece and at least part of the processing by said processing means takes place within the handpiece.

20. A device as claimed in claim 10 wherein said processing means uses the different scattering and absorption properties of different biological tissue as a reference in making said determination.

21. A device as claimed in claim 10 wherein said handpiece includes at least one user interface element and wherein said acoustic transducer assembly transmits acoustic energy in response to actuation of said at least one user interface element.

22. A device for assisting in positioning of a needle within a patient, said device comprising:
a handpiece for manipulation by a user;
a needle assembly mounted on the handpiece, said needle assembly comprising a needle including a needle tip and a central lumen;
an acoustic transducer device, disposed in or adjacent to said lumen, for, in use with the needle inserted in the patient, transmitting acoustic energy through said lumen so as to be emitted from the needle tip into the patent and receiving acoustic energy from the needle tip that is returned to the needle tip from a location within the patient;
processing means for processing the returned acoustic energy to provide a determination of the location within the patient at which the needle tip is positioned; and
a readout, connected to said processing means, for indicating to a user, based on said determination, a probability of the needle tip being located at a predetermined location in the patient.

23. A device as claimed in claim 22 wherein said readout comprises at least two different outputs indicating at least two different probabilities that the needle tip is located at said predetermined location.

24. A device as claimed in claim 23 wherein said predetermined location is a location within an intra-articular space within the patient.
